(19) United States
(12) Patent Application Publication
(10) Pub. No.: US 2009/0081714 A1
(43) Pub. Date: Mar. 26, 2009

(54) Assays

(75) Inventors: Leong Ng, Leicester (GB); Sarah Johnson, Bedfordshire (GB); Steve Eida, Cambs (GB); Paul Sheard, Northants (GB)

Correspondence Address: STEPTOE & JOHNSON LLP, 1330 CONNECTICUT AVENUE, N.W., WASHINGTON, DC, 20036, US

(73) Assignee: Inverness Medical Switzerland GMBH, Zug (CH)

(21) Appl. No.: 11/988,456

(22) PCT Filed: Jul. 10, 2006
(86) PCT No.: PCT/IB2006/002052

§ 371 Date: Apr. 30, 2008

Related U.S. Application Data
(60) Provisional application No. 60/697,550, filed on Jul. 11, 2005.

(51) Int. Cl. G01N 33/68; C07K 7/06; C07K 16/18

(52) U.S. Cl. 435/7.93; 435/7.1; 435/7.94; 530/328; 530/387.1

(57) ABSTRACT

Methods and assays for monitoring the cardiac health of a subject are provided. The method involves the detection of a urotensin surrogate in a sample leading to a more accurate and reliable diagnosis of early or late stages of decompensated heart failure, heart failure, risk of a heart failur in a subject than the measurement of the cyclic peptide of urotensin alone.

### TECHNICAL FIELD

The present invention relates to assays and methods for the diagnosis of heart failure in subjects.

### BACKGROUND

Heart failure is a chronic, progressive disease that affects 1.5-2% of the general population of the Western world. The prevalence and incidence of heart failure is growing due to an aging population. Heart failure occurs when the heart is not strong enough to pump blood efficiently around the body.

In many cases, the early stages of congestive heart failure are not recognized in a patient. Causes of heart failure can include acute cardiovascular events such as coronary artery disease, myocardial infarction (MI), stroke, hypertension and valvular heart disease (especially aortic and mitral disease). Other causes can include: drug toxicity, alcohol abuse, infections by viruses (including human immunodeficiency virus), bacteria and parasites; drugs (e.g., doxorubicin, cyclophosphamide, cocaine); alcohol; connective tissue disease; infiltrative disease (e.g., amyloidosis, sarcoidosis, hemochromatosis, malignancy); tachycardia; obstructive cardiomyopathy; peripartum cardiomyopathy; and dilated idiopathic cardiomyopathy.

The main contributing factor however is age: the heart weakens and blood vessels narrow with age. People over the age of 55 are most affected. Given an aging population, more people are expected to develop heart failure thus placing an ever increasing burden on healthcare. Heart failure is often not diagnosed until a patient presents with acute decompensation and an advanced stage of the disease. An ideal solution to the problem would be to diagnose heart failure or pre-heart failure earlier allowing therapeutic intervention and delaying disease progression. Further, diagnostic methods used in the acute setting have poor specificity. Another solution would be to provide the clinician with a more accurate method to diagnose heart failure in patients presenting with acute decompensation. Currently, there are no sensitive and specific methods to diagnose the onset of decompensation. Another solution would be to provide the patient or healthcare professional with an accurate method to detect the presence or start of decompensation thus avoiding acute decompensation.

### SUMMARY

A method for detecting a urotensin surrogate, such as, for example, prourotensin and prourotensin related peptide species, in a sample can be more accurate and reliable in diagnosing early or late stages of decompensated heart failure, heart failure, or risk of heart failure in a subject than measurement of the cyclic peptide of urotensin alone. Such assays may be used in combination with a second assay to detect a marker such as CRP, N-BNP, BNP, N-ANP, ANP, ORP150, myoglobin, troponin, creatine kinase MB or MPO to diagnose early or late stages of decompensated heart failure, heart failure, or risk of heart failure and to prognose adverse outcomes in subject. A subject with pre-heart failure or at risk for heart failure or heart failure or an early stage of decompensated heart failure can be managed in the home or a non-hospital setting. To help such subjects monitor the likelihood of cardiovascular events occurring or therapeutically manage their condition, a means is provided to detect or to monitor the subject's condition. A subject may include a human subject or a non-human subject such as birds, mice, rats, rabbits, pigs, cows, and other mammals.

In one aspect, a method for monitoring the cardiac health of a subject can include detecting a level of a urotensin surrogate in a sample from a subject. A subject may include a non-human subject. The sample can be blood or plasma or urine. The urotensin surrogate can be prourotensin or prourotensin related peptide. The level of urotensin surrogate can be detected by contacting a sample obtained from a subject with for example, an antibody that binds a urotensin surrogate. The antibody can bind to a peptide including at least a portion of SEQ ID NO: 1 and can bind specifically to the urotensin surrogate. The antibody can bind to a peptide including at least a portion of SEQ ID NO: 2 and can bind specifically to the urotensin surrogate. The antibody can be a monoclonal antibody or a polyclonal antibody. The level of urotensin surrogate can be determined with a competitive assay or a sandwich assay. The level of urotensin surrogate can be associated with heart failure.

The method for monitoring the cardiac health of a subject can include detecting a level of a urotensin surrogate in a sample from a subject and associating the level of the urotensin surrogate with the status of cardiac health. A subject may include a non-human subject. Associating the level of the urotensin surrogate with the status of cardiac health can include evaluating a risk of heart failure, monitoring an effect of therapy administered to the subject, screening for heart failure, diagnosing heart failure, determining heart failure or identifying a subject at risk for heart failure.

The method for monitoring the cardiac health of a subject can further include measuring the level of a marker in the sample and associating a level of the second marker with the status of cardiac health. The marker can include brain natriuretic peptide, atrial natriuretic peptide, C-type natriuretic peptide, troponin, creatine kinase MB isoform, ORP150, myeloperoxidase or myoglobin.

In another aspect, a method of assaying a sample for urotensin can include detecting a level of a urotensin surrogate in a sample from a subject. The sample can be blood or plasma or urine. The urotensin surrogate can be prourotensin or prourotensin related peptide. The level of urotensin surrogate can be detected by contacting a sample obtained from a subject with for example, an antibody that binds a urotensin surrogate. The antibody can bind to a peptide including at least a portion of SEQ ID NO: 1 and can bind specifically to the urotensin surrogate. The antibody can bind to a peptide including at least a portion of SEQ ID NO: 2 and can bind specifically to the urotensin surrogate. The antibody can be a monoclonal antibody or a polyclonal antibody. The level of urotensin surrogate can be determined with a competitive assay or a sandwich assay. The level of urotensin surrogate can be associated with heart failure.

In one aspect, a polypeptide includes the sequence: -EDARCTPEEL-. The sequence can include a substitution. The substitution can be a conservative substitution. In a further aspect, an antibody can be derived from a mammal immunized with a synthetic polypeptide and the polypeptide can include the sequence: -EDARCTPEELC-. The sequence can include a substitution. The substitution can be a conservative substitution. The antibody can be a polyclonal antibody or a monoclonal antibody. The antibody can recognize at least a portion of SEQ ID NO: 4. The antibody can recognize at least a portion of SEQ ID NO: 5.

In another aspect, a method of detecting a urotensin surrogate in a sample can include contacting the sample with an antibody having affinity for at least a portion of amino acids 1-113 of SEQ ID NO: 1. The antibody can have an affinity for a polypeptide which includes the sequence: -EDARCTPEEL-. The antibody can be derived from a mammal immunized with prourotensin. The antibody can be derived from a mammal immunized with a polypeptide comprising the sequence: -EDARCTPEELC-. The polypeptide can be a synthetic polypeptide. The antibody can be a polyclonal antibody or a monoclonal antibody.

In another aspect, an antibody can be derived from a mammal immunized with a synthetic polypeptide, the polypeptide comprising the sequence: -LSHLLARIWIUY-. In another aspect, an antibody can be derived from a mammal immunized with a synthetic polypeptide, the polypeptide comprising the sequence: -PLLDSREISFQ-. In a further aspect, an antibody can be derived from a mammal immunized with a synthetic polypeptide, the polypeptide comprising the sequence: -QLEKLKEQLVEEK-. In another aspect, an antibody can be derived from a mammal immunized with a synthetic polypeptide, the polypeptide comprising the sequence: -PDKKYTNREE-.

The method of detecting a urotensin surrogate in a sample can also include contacting the sample with an antibody having affinity for at least a portion of SEQ ID NO: 2 or an antibody having affinity for at least a portion of SEQ ID NO: 3, or an antibody having affinity for at least a portion of SEQ ID NO: 4 or an antibody having affinity for at least a portion of SEQ ID NO: 5 or an antibody having affinity for at least a portion of a linear peptide having the sequence of SEQ ID NO: 6.

In another aspect, a kit for monitoring the cardiac health of a subject can include instructions for taking a sample from the subject, and one or more reagents for measuring the level of a urotensin surrogate in the sample. The urotensin surrogate can be prourotensin or prourotensin related peptide. The reagents can include an antibody that binds specifically to prourotensin. The antibody can be a polyclonal antibody or a monoclonal antibody. The reagents can include an antibody that binds specifically to prourotensin related peptide. The antibody can be a polyclonal antibody or a monoclonal antibody. The kit can include one or more reagents for measuring the level of a second marker indicative of status of cardiac health. The second marker can include brain natriuretic peptide, atrial natriuretic peptide, C-type natriuretic peptide, troponin, creatine kinase MB isoform, ORP150, myeloperoxidase or myoglobin.

The instructions can include associating the level of the urotensin surrogate with the status of cardiac health. Associating the level of the urotensin surrogate with the status of cardiac health includes evaluating a risk of heart failure, monitoring an effect of therapy administered to the subject, screening for heart failure, diagnosing heart failure, determining heart failure or identifying a subject at risk for heart failure.

Many of the tests and procedures for accurately and successfully monitoring, diagnosing, managing and treating heart failure are complex, expensive and available only at a hospital or other health-care settings. Methods for patients to manage or to monitor the likelihood of heart failure at home or otherwise outside a health-care setting can be even less successful.

Most commercially available antibodies for urotensin, for example the Penlabs antibody, recognize the common ring structure and do not distinguish urotensin from urotensin related peptide. Further, results from immunoassays using antibodies against the cyclic peptide of urotensin (EP 1241479 A2) show that such antibodies give rise to irreproducible data and to unacceptably wide variations in experimental results. Those antibodies also failed to detect the different stages of heart failure and failed to diagnose heart failure patients from control healthy patients. In addition, most assays currently require urotensin to be extracted from the sample prior to measurement in order to remove interfering substances and require significant sample processing. Poor specificity coupled with lengthy processing time makes such assays unsuitable for routine use.

Advantageously, unique epitopes on the prourotensin or prourotensin related peptide provide unique and highly specific targets for antibody recognition. Such antibodies remove the requirement for extensive sample-pretreatment and provide immunoassays with higher sensitivity and specificity than has been achieved with antibodies that claim to be specific to urotensin.

The level of a urotensin surrogate, such as, for example, prourotensin or prourotensin related peptide, in a sample of bodily fluid from a subject is diagnostic of heart failure and can be diagnostic or prognostic of the condition of the subject. In particular, a level of prourotensin or prourotensin related peptide can be used as an initial screen with optional subsequent testing with a second marker to more efficiently diagnose heart failure, detect a worsening of heart failure, detect deteriorating heart function, or predict the likelihood of sudden unexpected death or response to a therapy in a subject. Urotensin surrogates can be diagnostic for heart failure in a subject with or without other manifestations of heart disease thus providing a means to screen for heart failure. Advantageously, heart failure can be identified or tracked by monitoring the level of a urotensin surrogate in a sample from a patient using antibodies that specifically bind to such a urotensin surrogate.

In one embodiment, a urotensin surrogate in combination with BNP can be useful or a more sensitive means for monitoring the cardiac health of a subject. Currently, assays for monitoring heart failure in a subject using the BNP marker has low specificity, low sensitivity, and are not cost-effective as many patients still have to be referred for echocardiography due to unconclusive test results. Levels of BNP are not always increased in decompensated heart failure thus limiting BNP's utility as a marker of decompensation in a patient. Further, levels of BNP can vary with a number of physiological factors such as, for example, age and obesity. A urotensin surrogate is more reliable in the prediction of heart failure as concentrations of urotensin surrogate are not linked to physiological factors such as obesity, or renal function.

The details of one or more embodiments are set forth in the accompanying drawings and the description below. Other features, objects, and advantages will be apparent from the description and drawings, and from the claims.

### BRIEF DESCRIPTION OF THE DRAWING

FIG. 1 shows the amino acid sequence for human prourotensin (SEQ ID NO: 1).

FIG. 2A shows the amino acid sequence for human prourotensin related peptide (SEQ ID NO: 2).

FIG. 3A is a graph depicting standard assay curves of various antibodies directed against the prourotensin epitope EDARCTPEEL.

FIG. 3B is a graph depicting the difference in prourotensin levels between normal and heart failure patients using antibody 7519 (anti-prourotensin).

### DETAILED DESCRIPTION

Recent work has revealed the existence of a novel cardiovascular peptide called urotensin II with homology to the hormone of teleosts (Ames et al., Nature 1999; 16: 282-286). This cyclic undecapeptide is the ligand for an orphan G-protein receptor (GPR14) and both peptide and receptors are distributed within the myocardium, endothelium, vascular myocytes and nervous system (Ames et al., Nature 1999; 16: 282-286). Research by Douglas et al (Douglas et al. Lancet 2002; 359: 1990-1997) examined the expression of urotensin and its receptor, GPR14 (or UT receptor) in myocardium of patients at various stages of congestive heart failure. Urotensin was found to be strongly expressed in cardiomyocytes, vascular smooth muscle cells, endothelium and inflammatory cells within the myocardium of patients with congestive heart failure.

Urotensin has been detected in assays of plasma at higher levels in patients who have heart failure than normal subjects. See, for example, U.S. Patent Application Nos. 20040077027 and 20050014198.

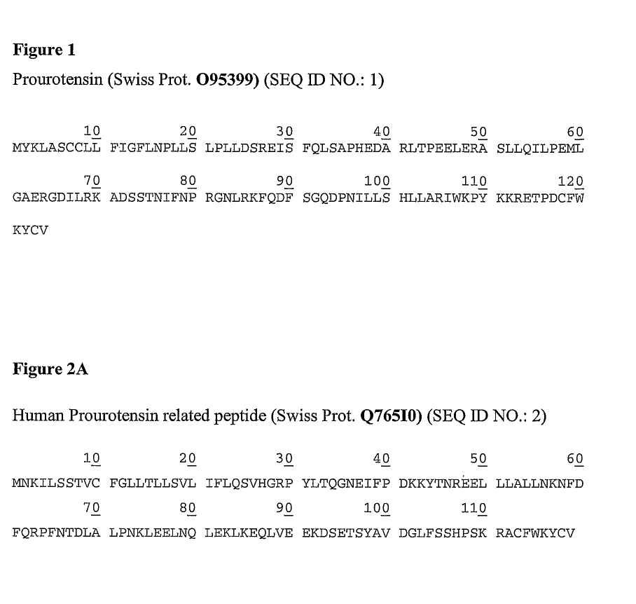

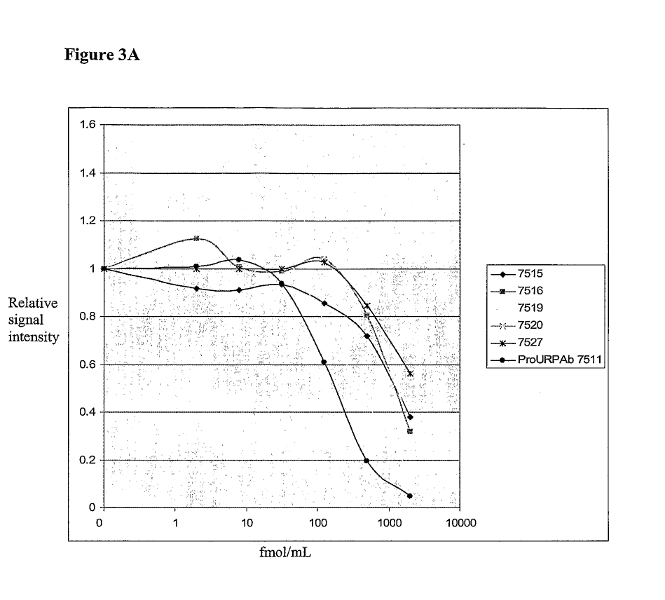

Urotensin (see, for example, GenBank Accession Numbers NM_{—}021995 and NM_{—}006786) is derived from a prohormone precursor, prourotensin (see, for example, GenBank Accession Number 095399, UniProtKB/SwissPrott entry 095399, FIG. 1), which is processed to mature urotensin and an N-terminal peptide. Another form of urotensin includes the urotensin related peptide (URP) which is derived from prourotensin related peptide (see, for example, GenBank Accession Number NM_{—}198152, UniProtKB/SwissPrott entry Q76510 (human URP), FIG. 2). The mature urotensin and urotensin related peptide share a common ring structure (Cys-Phe-Trp-Lys-Tyr-Cys-Val) with disuphide bonds between the Cys. The mature urotensin and urotensin related peptides differ in their amino-terminal region of the mature peptides. The amino terminal sequence of mature urotensin begins with Glu-Thr-Pro-Asp whereas the amino terminal sequence of mature prourotensin related peptide begins with Ala.

Advantageously, the measurement of a urotensin surrogate can be useful as a more sensitive and accurate means for detecting heart failure and for assessing the severity of heart failure. Urotensin surrogate is a polypeptide associated with urotensin, for example, a peptide whose presence is correlated with the presence of urotensin. Such a polypeptide does not include the cyclic form of urotensin, i.e. the cyclic form of SEQ ID. NO. 6). Urotensin surrogate may include unprocessed full length prourotensin or prourotensin related peptide (with the hormone still attached), fragments or variants thereof (e.g., allelic variants). Urotensin surrogate may further include the potential circulating forms of prourotensin such as amino acids 21-113 of prourotensin (SEQ ID NO.:3), amino acids 21-110 of prourotensin (SEQ ID NO.:4), amino acids 21-124 of prourotensin (SEQ ID NO.:5) or fragments and variants thereof. Urotensin surrogate may also include linearized urotensin (SEQ ID NO.:6).

| | |
|---|---|
| SEQ ID NO.:3 | LPLLDSREIS FQLSAPHEDA RLTPEELERA |
| | SLLQILPEML GAERGDILRK ADSSTNIFNP |
| | RGNLRKFQDF SGQDPNILLS HLLAPIWKPY |
| | KKR |
| SEQ ID NO.:4 | LPLLDSREIS FQLSAPHEDA RLTPEELERA |
| | SLLQILPEML GAERGDILRK ADSSTNIFNP |
| | RGNLRKFQDF SGQDPNILLS HLLARIWKPY |
| SEQ ID NO.:5 | LPLLDSREIS FQLSAPHEDA RLTPEELERA |
| | SLLQILPEML GAERGDILRK ADSSTNIFNP |
| | RGNLRKFQDF SGQDPNILLS HLLARIWKPY |
| | KKRETPDCFW KYCV |
| SEQ ID NO.:6 | ETPDCFW KYCV |

Measurement of the urotensin surrogate can be useful for monitoring the cardiac health of a subject. The urotensin surrogate in combination with a second marker such as C-reactive protein (CRP), myeloperoxidase (MPO) or natriuretic peptides such as A-type- (ANP), B-type- (BNP), and C-type- (CNP) natriuretic peptide and their N-terminal prohormones (N-ANP, N-BNP, and N-CNP) can be used to detect heart failure in patients who are as yet undiagnosed, to detect volume overload (decompensation) in a heart failure patient, to determine whether a patient's condition is worsening or improving (for example changing NYHA Class). NYHA classification refers to the New York Heart Association (NYHA) classification. This is a four-stage classification where class 1 patients exhibit symptoms only at exertion levels, class 2 patients exhibit symptoms with ordinary exertion, class 3. Patients exhibit symptoms with minimal exertion and class 4 patients exhibit symptoms at rest. The levels of urotensin surrogate can be used to identify the different classes of heart failure in a patient.

The urotensin surrogate in combination with a second marker can also be used to determine whether a heart failure patient's condition is responding to therapy, as a risk factor for developing heart failure in individuals with Stage A (risk factors relevant to heart failure) or Stage B (pre-heart failure, e.g. hypertension), and/or as a risk factor for developing sudden cardiac death, e.g. it could be used with other markers and a measure of heart rate variability.

A method of evaluating the levels of urotensin surrogate can include observing a change in intraindividual variability, observing a change in absolute or relative level from a baseline level established within an individual or observing a positive or negative trend over time. In another aspect, a cardiac health of a subject can be monitored by measurement of urotensin surrogates levels in combination with measurement of a patient's vital signs, such as weight, temperature, heart rate, breathing rate, blood pressure, and blood oxygen saturation (pulse oximetry). The levels of urotensin surrogate may also be measured in combination with chest X-ray, electrocardiography, echocardiography, radionuclide imaging and dilutional analysis.

A level of a urotensin surrogate in a sample can be measured by quantifying the amount of the marker in the sample as a whole molecule, as fragments of the marker, or by measuring the marker's activity in the sample or a derivative of the sample. The level of a urotensin surrogate in a sample may be measured by quantifying the amount of the marker in the sample as a linear form or as a ring form. To measure a urotensin surrogate in a linear form, the sample will undergo a reduction step prior to analysis.

Fragments of the above-described markers can be measured using a fragment that will have an amino acid sequence which is unique to the marker in question. The fragment may be as few as 6 amino acids, although it may be 7, 8, 9, 10, 11, 12, 13, 14, 15 or more amino acids.

The structure of prourotensin or prourotensin related peptide can be described with a polypeptide sequence. For example, the structure can be described by a single polypeptide sequence representing the full polypeptide sequence of prourotensin or prourotensin related peptide, or the structure can be described by a partial sequence, corresponding to a fragment of prourotensin or prourotensin related peptide. A plurality of partial sequences can be combined to make a larger partial sequence or the full sequence. For example, a first polypeptide sequence can represent the N-terminal polypeptide sequence of prourotensin or prourotensin related peptide, and a second sequence can represent the C-terminal polypeptide sequence of prourotensin or prourotensin related peptide. An artificial polypeptide sequence of prourotensin can include:

-EDARCTPEELC-
-LSHLLARIWKPY-
-PLLDSREISFQ-

A polypeptide sequence of prourotensin related peptide can include:

-QLEKLKEQLVEEK-
-PDKKYTNREE-

Where polypeptide sequences are listed with a dash (“−”) at one or both ends, the dash indicates a terminus, or an additional amino acid or peptide sequence occurring N-terminal or C-terminal to the sequence presented. The N- or C-terminus can be modified, such as with a formyl group on the N-terminus. The C-terminus can be modified with the addition of a Cys residue for conjugation purposes. The additional peptide sequence can be modified, (for example, glycosylated, phosphorylated, biotinylated, modified with a hydrophobic group (e.g., myristoylated or geranylgeranylated), or other peptide modification. If the polypeptide is synthetic, the modification can include, for example, a colored or fluorescent group, or a poly(ethylene glycol) group.

In general, an amino acid residue of the polypeptide can be replaced by another amino acid residue in a conservative substitution. Examples of conservative substitutions include, for example, the substitution of one non-polar (i.e., hydrophobic) residue such as isoleucine, valine, leucine or methionine for another non-polar residue; the substitution of one polar (i.e. hydrophilic) residue for another polar residue, such as a substitution between arginine and lysine, between glutamine and asparagine, or between glycine and serine; the substitution of one basic residue such as lysine, arginine or histidine for another basic residue; or the substitution of one acidic residue, such as aspartic acid or glutamic acid for another acidic residue. In an conservative substitution, an amino acid residue can be replaced with an amino acid residue having a chemically similar side chain. Families of amino acid residues having side chains with chemical similarity have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A conservative substitution may also include the use of a chemically derivatized residue in place of a non-derivatized residue. A chemical derivative residue is a residue chemically derivatized by reaction of a functional group of the residue. Examples of such chemical derivatives include, but are not limited to, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters, or other types of esters or hydrazides. Free hydroxyl groups may be derivatized to form O-acyl or O-alkyl derivatives. Also included as chemical derivatives are those polypeptides which contain one or more naturally-occurring amino acid derivatives of the twenty standard amino acids. For example, 4-hydroxyproline may be substituted for proline; 5-hydroxylsine may be substituted for histidine; homoserine may be substituted for serine; and ornithine may be substituted for lysine.

An amino acid residue of the polypeptide can be replaced by another amino acid residue in a non-conservative substitution. In some cases, a non-conservative substitution will not alter the relevant properties of the polypeptide. The relevant properties can be, without limitation, ability to bind to an antibody that recognizes urotensin surrogates such as prourotensin, prourotensin related peptide or other biological activity.

An antibody is an immunoglobulin molecule or an immunologically active portion of an immunoglobulin molecule, i.e., a molecule that contains an antigen binding site that specifically binds an antigen. The immunoglobulin molecules can be of any class (e.g., IgG, IgE, IgM, IgD and IgA) or subclass of immunoglobulin molecule. Antibodies includes, but are not limited to, polyclonal, monoclonal, bispecific, humanised and chimeric antibodies, single chain antibodies, Fab fragments and F(ab′)2 fragments, fragments produced by a Fab expression library, anti idiotypic (anti Id) antibodies, and epitope binding fragments of any of the above. An antibody, or generally any molecule, binds specifically to or has affinity for an antigen (or other molecule) if the antibody binds preferentially to the antigen, and, e.g., has less than about 30%, preferably 20%, 10%, or 1% cross-reactivity with another molecule. Portions of antibodies include Fv and Fv′ portions.

Antibodies prepared against a peptide may be tested for activity against that peptide as well as the full length protein. Antibodies may have affinities of at least about 10⁻⁶M, 10⁻⁷M, 10⁻⁸M, 10⁻⁹M, 10⁻¹⁰M, 10⁻¹¹M or 10⁻¹²M toward the peptide and/or the full length polypeptide.

In general, polyclonal antibodies that recognize a particular polypeptide can be generated by immunizing a mammal (such as a mouse or rabbit) with the polypeptide. The polypeptide can be prourotensin or prourotensin related peptide, a fragment or cleavage product of prourotensin or prourotensin related peptide, or a prourotensin or prourotensin related peptide analog. The polypeptide can include other sequences besides a urotensin surrogate sequence. For example, the prourotensin or prourotensin related peptide analog can be biologically active (for example, sharing some or all of the biological effects of prourotensin or prourotensin related peptide) or biologically inactive. Whether biologically active or inactive, the analog can serve as an antigen for generating antibodies that recognize for example, prourotensin or prourotensin related peptide. The polypeptide antigen can be linked to a larger polypeptide by chemical methods, or cloned and expressed as a fusion with a larger polypeptide. The polypeptide antigen can be injected as a mixture with an adjuvant, such as Freund's complete adjuvant. An ELISA assay can be used to determine the titer of antibodies in serum collected from the animal. Detailed procedures for the generation of polyclonal antibodies can be found, for example, in Current Protocols in Immunology, 2001, John E. Coligan, ed., John Wiley & Sons.

In general, monoclonal antibodies that recognize a particular polypeptide can be generated by immunizing a BALB/c mouse with the polypeptide. The polypeptide can be linked to a larger polypeptide by chemical methods, or cloned and expressed as a fusion with a larger polypeptide. The polypeptide antigen can be injected as a mixture with an adjuvant, such as Freund's complete adjuvant. Spleen cells from the immunized mouse can be fused with myeloma cells to form immortal, antibody-expressing cells. Cells that express an antibody having specificity for the desired polypeptide can be isolated and used to produce additional quantities of the monoclonal antibody. Detailed procedures for the generation of monoclonal antibodies can be found, for example, in Current Protocols in Immunology, 2001, John E. Coligan, ed., John Wiley & Sons.

When an antibody is made by the methods described above, it can be described as being derived from a mammal (for example, a mouse or rabbit in the description above). A monoclonal antibody produced from a hybridoma cell culture is considered to be derived from the mammal, since the hybridoma is made by fusing cells from the mammal immunized with an antigen.

The level of urotensin surrogate in a sample can be measured qualitatively or quantitatively using an assay, for example, in an immunochromatographic format. A qualitative assay can be distinguish between the presence or absence of a marker, or can distinguish between categories of marker levels in a sample, such as absent, low concentration, medium concentration or high concentration, or combinations thereof. A quantitative assay can provide a numerical measure of a marker in a sample. The assay can include contacting a marker with an antibody that recognizes that particular marker, detecting the marker by mass spectrometry, assaying a sample including cells for expression (e.g., of mRNA or polypeptide) of the marker gene by the cells, or a combination of measurements. For example, the assay can include contacting a sample with an antibody that recognizes the marker and a mass spectrometry measurement.

The urotensin surrogate can be detected in blood, plasma or other bodily fluids which can be obtained from a body, such as interstitial fluid, urine, whole blood, saliva, serum, lymph, gastric juices, bile, sweat, tear fluid and brain and spinal fluids. Bodily fluids may be processed (e.g. serum) or unprocessed.

A second marker indicative of heart failure in a subject can also be measured. The second marker can be C-reactive protein (CRP—SwissProt P02741), Oxygen Regulated Protein (ORP150—NCBI database Accession AAC50947, Accession NP_{—}006380), myeloperoxidase (NCBI database Accession NM_{—}000250, Accession AH_{—}002972), Plasma Surfactant Protein-B (GenBank Accession Number J02761), Nourin-1 (as described in patent application Ser. No. 10/945,442) or a natriuretic peptide, including a native atrial natriuretic peptide (ANP—see Brenner et al, Physiol. Rev., 1990, 70: 665), brain natriuretic peptide (BNP) and C-type natriuretic (CNP— see Stingo et al, Am. J. Physiol. 1992, 263: H1318), and variants or allelic variants thereof. A suitable natriuretic peptide is brain natriuretic peptide (BNP) or N-terminal pro-brain natriuretic peptide (N-BNP). In heart failure, there is evidence of upregulation of the Brain natriuretic peptide system, with increased plasma levels of brain natriuretic peptide (BNP) (Wei et al, Circulation 1993; 88: 1004-9; McDonagh et al, Lancet 1998; 351: 9-13), and its precursor N-terminal protein (N-BNP) (Hunt et al, Clin Endocrinol 1997; 47: 287-96; Hughes et al, Clinical Science 1999; 96: 373-380). The whole protein (Sudoh et al, Biochem Biophys Res Commun 1989; 159: 1427-34) consists of a signal peptide sequence (amino acids 1-26), and proBNP (amino acids 27-134), from which is derived the N-BNP (amino acids 27-102) and BNP (amino acids 103-134). The release of proBNP (the intact precursor to the two circulating forms, BNP (the active peptide) and N-BNP (the inactive peptide)) from cardiac myocytes in the left ventricle and increased production of BNP is triggered by myocardial stretch, myocardial tension, and myocardial injury. Another suitable natriuretic peptide is atrial natriuretic peptide (ANP) (Hall, Eur J Heart Fail, 2001, 3:395-397). The use of Plasma Surfactant Protein-B as a marker of heart failure is described in De Pasquale et al, Circulation, 2004, 110:1091-1096.

Fragments of the above-described second markers can be measured. In this case, the fragment will have an amino acid sequence which is unique to the second marker in question. The fragment may be as few as 6 amino acids, although it may be 7, 8, 9, 10, 11, 12, 13, 14, 15 or more amino acids. For ORP150, the fragment may include the sequence LAVMSVDLGSESM. For BNP, the fragment may include the sequence PQTAPSRALLLLL.

Marker levels may be provided in units of concentration, mass, moles, volume or any other measure indicating the amount of marker present.

The respective levels of the urotensin surrogate and secondary markers may be measured using an immunoassay, for example, by contacting the sample with an antibody that binds specifically to the marker and measuring any binding that has occurred between the antibody and at least one species in the sample. Such assays may be competitive or non-competitive immunoassays. The assay can be homogeneous or heterogeneous. The analyte to be detected can be caused to bind with a specific binding partner such as an antibody which has been labelled with a detectable species such as a latex or gold particle, a fluorescent moiety, an enzyme, an electrochemically active species, etc. Alternatively, the analyte can be labelled with any of the above detectable species and competed with limiting amounts of specific antibody. The presence or amount of analyte present is then determined by detection of the presence or concentration of the label. Such assays may be carried out in the conventional way using a laboratory analyser or with point of care or home testing device, such as the lateral flow immunoassay as described in EP291194.

In one embodiment, an immunoassay is performed by contacting a sample from a subject to be tested with an appropriate antibody under conditions such that immunospecific binding can occur if the marker is present, and detecting or measuring the amount of any immunospecific binding by the antibody. The antibody may be contacted with the sample for at least about 10 minutes, 30 minutes, 1 hour, 3 hours, 5 hours, 7 hours, 10 hours, 15 hours, or 1 day. Any suitable immunoassay can be used, including, without limitation, competitive and non competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays and protein A immunoassays.

In another embodiment, antibodies to urotensin surrogate can be immobilized on a surface in a sandwich assay. A sample of interest is allowed to interact with the immobilized antibodies. If urotensin surrogate is present in the sample, it will be bound by the antibodies and thus become immobilized. After incubation, the surface can be washed prior to addition of a second antibody to urotensin surrogate. The second antibody can recognize a different epitope of urotensin surrogate than the immobilized antibody. The second antibody may recognize urotensin surrogate which may include the ring structure. The sandwich assay may further include first and second antibodies that recognize either linear or ring forms of urotensin surrogate.

If urotensin surrogate was present in the initial sample, an immobilized antibody/urotensin surrogate/second antibody sandwich forms. The second antibody can be coupled to a colored material, or alternatively, the sandwich can then be detected by a third antibody. Typically the third antibody is an anti-IgG antibody derived from a different species than the second antibody. For example, if the second antibody to prourotensin or prourotensin related peptide is a mouse IgG, then the third antibody can be a goat anti-mouse IgG antibody or a rabbit anti-mouse IgG antibody.

The second or third antibody can produce a detectable change when bound to its target. For ease of detection of the sandwich, the second or third antibody can be associated with a color-developing reagent. The color-developing reagent can be a colored material (such as a dye or colored latex particle) or a reagent capable of converting a colorless material to a colored material. One such reagent is a peroxidase enzyme linked to the third antibody. In the presence of appropriate substrates, the peroxidase enzyme can produce a colored product, which is easily detected by virtue of its color. The use of an enzyme (or other catalyst) to produce a detectable change in samples having urotensin surrogate can increase the sensitivity of the assay. Other methods of detecting an antigen (such as prourotensin or prourotensin related peptide) using antibodies to the antigen are known. Examples of immunochromatographic tests and test result readers can be found in, for example, U.S. Pat. Nos. 5,504,013; 5,622,871; 6,235,241; and 6,399,398.

Another method of detecting urotensin surrogate can include the use of a ligand. A ligand can include, for example, a modified antibody, chimeric antibody, soluble receptor, aptamer, or other species capable of binding to urotensin surrogate. An aptamer is a single- or double-stranded DNA or single-stranded RNA molecules that recognize and bind to a desired target molecule by virtue of their shapes. See, e.g., PCT Publication Nos. WO 92/14843, WO 91/19813, and WO 92/05285. The ligand can be detectably labeled, for example with a fluorescent dye, colored material, or radioactive isotope.

### EXAMPLES

A peptide from the prosequence of urotensin (EDARCTPEEL-cysteine (38-47) conjugated to a carrier molecule was used as an immunogen to create monoclonal antibodies. BALB/c mice were immunized initially via intraperitoneal injections. Mouse serum was obtained then days after the second injection and then tested for activity. The mouse which had serum that exhibited the highest possible anti-prourotensin activity was chosen for cell fusion. Spleens were collected and cell suspensions were prepared by perfusion with Dulbecco's Modified Eagle Medium (DMEM). Spleen cell suspension containing B-lymphocytes and macrophages was prepared by perfusion of the spleen. The cell suspension was washed and collected by centrifugation; myeloma cells were also washed in this manner. Live cells were counted and the cells placed into a 37 degree C. water bath. One mL of 50% polyethylene glycol (PEG) was added slowly to DMEM. The BLAB/c spleen cells were fused with SP 2/0-Ag 14 mouse myeloma cells by PEG and the resultant hybridomas were grown in hypoxanthine (H), aminopterin (A) and thymidine (T) (HAT) selected tissue culture media plus 20% fetal calf serum. The surviving cells were allowed to grow to confluence. The spent culture medium was checked for antibody titer, specificity, and affinity. The cells were incubated in the PEG for one to 1.5 minutes at 37 degree C., after which the PEG was diluted by the slow addition of DMEM media. The cells were pelleted and 35 to 40 mL of DMEM containing 10% fetal bovine serum was added. The cells were then dispensed into tissue culture plates and incubated overnight in a 37 degree C., 5% CO₂, humidified incubator. DMEM-FCS containing hypoxanthine (H), aminopterin (A) and thymidine (T) medium (HAT medium) was added to each well. Subsequently, the plates were incubated with HAT medium every three to four days for two weeks. Fused cells were then grown in DMEM-FCS containing HAT medium. As cell growth became ½ to ¾ confluent on the bottom of the wells, supernatant tissue culture fluid was taken and tested for prourotensin specific antibody. Positive wells were cloned by limiting dilution over macrophage or thymocyte feeder plates, and cultured in DMEM-FCS. Cloned wells were tested and recloned three times before a statistically significant monoclonal antibody was obtained. Spent culture media from the chosen clone contained antibody which bound prourotensin in all dilutions tested.

Peptides to which the antibodies were raised were coated onto the surface of 96 well microtitre plates, by incubating in carbonate buffer overnight at 4° C. Plates were then blocked with a non-specific protein such as bovine serum albumin or casein for 20 minutes at room temperature. Antibodies were serially diluted, then applied to the coated plate for 1 hour at room temperature. The bound antibody was then detected using an anti-mouse IgG coupled to an enzyme, such as horseradish peroxidase or alkaline phosphatase. Appropriate enzyme substrates, such as 3,3,5,5-tetramethylbenzidene (TMB), 5-bromo-4-chloro-3-indoxyl phosphate (BCIP) or dinitrophenyl phosphate (dNPP) were then used to detect binding.

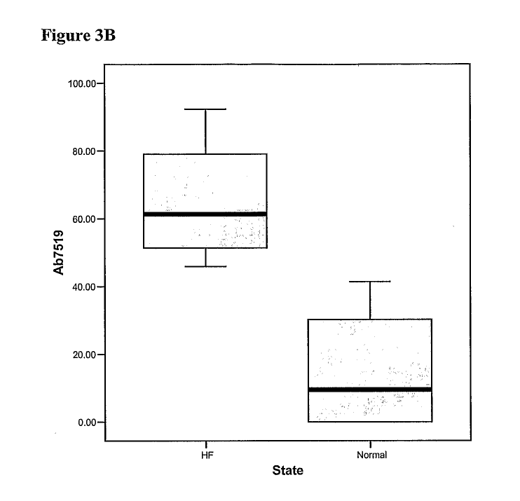

To obtain assay curves, 10 ng of antibody was incubated with standard at the appropriate concentration overnight at 4° C. The antibody was further incubated with 200 fmol of tracer-biotinylated proUrotensin peptide (EDARCTPEEL-cysteine-biotin). Immunoprecipitates were recovered in anti-rabbit IgG coated ELISA plases. After washes and incubation with methyl-acridinium ester (MAE)-labeled streptavidin, chemiluminescence was elicited. FIG. 3A illustrates standard assay curves obtained with various antibodies directed against the prourotensin epitope EDARCTPEEL. The x-axis gives concentration of prourotensin peptide (EDARCTPEEL) fmol/ml. The y-axis indicates relative signal intensity, where the signal at the 0 standard was assigned a relative intensity of 1, and all further standards were expressed as a proportion of this.

An assay was set up using a preparation of the antibody (tissue culture supernatant) and labelled peptide and was used to show a difference between normal and heart failure patients (see FIG. 3B).

Plasma samples from four heart failure patients and four normal volunteers were collected into EDTA plasma vacutainers. The plasma was separated immediately on receipt and kept frozen at −80° C. until analysis. Prior to analysis the prourotensin was extracted from the plasma on a C18 column. Columns were primed by washing with 3 ml of methanol, followed by 2×3 ml washes with 1% TFA (trifluoroacetic acid). The plasma sample (1 ml) was then applied to the column and allowed to drip through. Unbound material was washed from the column with 3 ml of 1% TFA. Interfering substances were removed by washing the bound peptides with 3 ml of 20% acetonitrile/1% TFA. The bound prourotensin and prourotensin related peptides were then eluted from the column by adding 3 ml of 60% acetonitrile/1% TFA. The eluted peptides were then lyophilised and reconstituted in water. The prourotensin present in the samples was measured with prourotensin antibody 7519 using the method described previously.

Each and every reference cited herein is hereby incorporated in its entirety for all purposes to the same extent as if each reference were individually incorporated by reference. Furthermore, while the invention has been described in detail with reference to preferred embodiments thereof, it will be apparent to one skilled in the art that various changes can be made, and equivalents employed, without departing from the scope of the invention.

1. A method for monitoring the cardiac health of a subject comprising detecting a level of a urotensin surrogate in a sample from a subject.

2. The method of claim 1, wherein the subject is a non-human subject.

3. The method of claim 1, wherein the sample is blood or plasma or urine.

4. The method of claim 1, wherein the urotensin surrogate is prourotensin.

5. The method of claim 1, wherein the urotensin surrogate is prourotensin related peptide.

6. The method of claim 1, wherein the level of urotensin surrogate is detected by contacting a sample obtained from a subject with an antibody that binds a urotensin surrogate.

7. The method of claim 1, wherein the level of urotensin surrogate is associated with heart failure.

8. The method of claim 6, wherein the antibody binds to a peptide including at least a portion of SEQ ID NO: 1 and binds specifically to the urotensin surrogate.

9. The method of claim 6, wherein the antibody binds to a peptide including at least a portion of SEQ ID NO: 2 and binds specifically to the urotensin surrogate.

10. The method of claim 6, wherein the antibody is a monoclonal antibody.

11. The method of claim 6, wherein the antibody is a polyclonal antibody.

12. The method of claim 1, wherein the level of urotensin surrogate is determined with a competitive assay.

13. The method of claim 1, wherein the level of urotensin surrogate is determined with a sandwich assay.

14. The method of claim 1, wherein detecting the level of the urotensin surrogate includes associating the level of the urotensin surrogate with the status of cardiac health.

15. The method of claim 14, wherein associating the level of the urotensin surrogate with the status of cardiac health includes evaluating a risk of heart failure.

16. The method of claim 14, wherein associating the level of the urotensin surrogate with the status of cardiac health includes monitoring an effect of therapy administered to the subject.

17. The method of claim 14, wherein associating the level of the urotensin surrogate with the status of cardiac health includes screening for heart failure.

18. The method of claim 14, wherein associating the level of the urotensin surrogate with the status of cardiac health includes diagnosing heart failure.

19. The method of claim 14, wherein associating the level of the urotensin surrogate with the status of cardiac health includes determining heart failure.

20. The method of claim 14, wherein associating the level of the urotensin surrogate with the status of cardiac health includes identifying a subject at risk for heart failure.

21. The method of claim 1, further comprising measuring the level of a marker in the sample and associating a level of the marker with the status of cardiac health.

22. The method of claim 21, wherein the second marker is brain natriuretic peptide, atrial natriuretic peptide, C-type natriuretic peptide, troponin, creatine kinase MB isoform, ORP150, myeloperoxidase or myoglobin.

23. A method of assaying a sample for urotensin comprising detecting a level of a urotensin surrogate in a sample from a subject.

24. The method of claim 23, wherein the subject is a non-human subject.

25. The method of claim 23, wherein the sample is blood or plasma or urine.

26. The method of claim 23, wherein the urotensin surrogate is prourotensin.

27. The method of claim 23, wherein the urotensin surrogate is prourotensin related peptide.

28. The method of claim 23, wherein the level of urotensin surrogate is detected by contacting a sample obtained from a subject with an antibody that binds a urotensin surrogate.

29. The method of claim 23, wherein the level of urotensin surrogate is associated with heart failure.

30. The method of claim 28, wherein the antibody binds to a peptide including at least a portion of SEQ ID NO: 1 and binds specifically to the urotensin surrogate.

31. The method of claim 28, wherein the antibody binds to a peptide including at least a portion of SEQ ID NO: 2 and binds specifically to the urotensin surrogate.

32. The method of claim 28, wherein the antibody is a monoclonal antibody.

33. The method of claim 28, wherein the antibody is a polyclonal antibody.

34. The method of claim 23, wherein the level of urotensin surrogate is determined with a competitive assay.

35. The method of claim 23, wherein the level of urotensin surrogate is determined with a sandwich assay.

36. A polypeptide comprising the sequence: -EDARCTPEEL-.

37-38. (canceled)

39. An antibody derived from a mammal immunized with a synthetic polypeptide, the polypeptide comprising the sequence: -EDARCTPEELC-.

40-45. (canceled)

46. A method of detecting a urotensin surrogate in a sample comprising contacting the sample with an antibody having affinity for at least a portion of amino acids 1-113 of SEQ ID NO: 1.

47-52. (canceled)

53. A kit for monitoring the cardiac health of a subject comprising:
instructions for taking a sample from the subject;
one or more reagents for measuring the level of a urotensin surrogate in the sample.

54-70. (canceled)

71. A method of detecting a urotensin surrogate in a sample comprising contacting the sample with an antibody having affinity for at least a portion of SEQ ID NO: 2, SEQ ID NO: 3, SEQ ID NO: 4, or SEQ ID NO: 5.

72-74. (canceled)

75. A method of detecting a urotensin surrogate in a sample comprising contacting the sample with an antibody having affinity for at least a portion of a linear peptide having the sequence of SEQ ID NO: 6.

76-79. (canceled)


SEQUENCE LISTING (16 sequences)

SEQ ID NO: 1 - protein, 124 aa, Homo sapiens
MYKLASCCLLFIGFLNPLLSLPLLDSREISFQLSAPHEDARLTPEELERASLLQILPEMLGAERGDILRKADSSTNIFNPRGNLRKFQDFSGQDPNILLSHLLARIWKPYKKRETPDCFWKYCV

SEQ ID NO: 2 - protein, 119 aa, Homo sapiens
MNKILSSTVCFGLLTLLSVLIFLQSVHGRPYLTQGNEIFPDKKYTNREELLLALLNKNFDFQRPFNTDLALPNKLEELNQLEKLKEQLVEEKDSETSYAVDGLFSSHPSKRACFWKYCV

SEQ ID NO: 3 - protein, 93 aa, Homo sapiens
LPLLDSREISFQLSAPHEDARLTPEELERASLLQILPEMLGAERGDILRKADSSTNIFNPRGNLRKFQDFSGQDPNILLSHLLARIWKPYKKR

SEQ ID NO: 4 - protein, 90 aa, Homo sapiens
LPLLDSREISFQLSAPHEDARLTPEELERASLLQILPEMLGAERGDILRKADSSTNIFNPRGNLRKFQDFSGQDPNILLSHLLARIWKPY

SEQ ID NO: 5 - protein, 104 aa, Homo sapiens
LPLLDSREISFQLSAPHEDARLTPEELERASLLQILPEMLGAERGDILRKADSSTNIFNPRGNLRKFQDFSGQDPNILLSHLLARIWKPYKKRETPDCFWKYCV

SEQ ID NO: 6 - protein, 11 aa, Homo sapiens
ETPDCFWKYCV

SEQ ID NO: 7 - protein, 10 aa, Artificial Sequence
  Description of Artificial Sequence Synthetic peptide
EDARCTPEEL

SEQ ID NO: 8 - protein, 11 aa, Artificial Sequence
  Description of Artificial Sequence Synthetic peptide
EDARCTPEELC

SEQ ID NO: 9 - protein, 12 aa, Artificial Sequence
  Description of Artificial Sequence Synthetic peptide
LSHLLARIWKPY

SEQ ID NO: 10 - protein, 11 aa, Artificial Sequence
  Description of Artificial Sequence Synthetic peptide
PLLDSREISFQ

SEQ ID NO: 11 - protein, 13 aa, Artificial Sequence
  Description of Artificial Sequence Synthetic peptide
QLEKLKEQLVEEK

SEQ ID NO: 12 - protein, 10 aa, Artificial Sequence
  Description of Artificial Sequence Synthetic peptide
PDKKYTNREE

SEQ ID NO: 13 - protein, 7 aa, Artificial Sequence
  Description of Artificial Sequence Synthetic peptide
CFWKYCV

SEQ ID NO: 14 - protein, 4 aa, Artificial Sequence
  Description of Artificial Sequence Synthetic peptide
ETPD

SEQ ID NO: 15 - protein, 13 aa, Artificial Sequence
  Description of Artificial Sequence Synthetic peptide
LAVMSVDLGSESM

SEQ ID NO: 16 - protein, 13 aa, Artificial Sequence
  Description of Artificial Sequence Synthetic peptide
PQTAPSRALLLLL